(19) United States
(12) Patent Application Publication
(10) Pub. No.: US 2023/0072265 A1
(43) Pub. Date: Mar. 9, 2023

(54) AN IMPROVED PROCESS FOR THE PREPARATION OF 4-({(1 R)-2-[5-(2-FLUORO-3METHOXYPHENYL)-3-{[2-FLUORO-6-(TRIFLUORO METHYL) PHENYL]METHYL}-4-METHYL-2,6-DIOXO-3,6DIHYDROPYRIMIDIN-1(2 H)-YL]-1-PHENYLETHYL}AMINO)BUTANOIC ACID OR ITS PHARMACEUTICALLY ACCEPTABLE SALTS

(71) Applicants: Thirumalai Rajan SRINIVASAN, Telangana, Hyderabad (IN); MSN LABORATORIES PRIVATE LIMITED, R&D CENTER, Sangareddy (Dist), Hyderabad, Telangan (IN)

(72) Inventors: Thirumalai Rajan SRINIVASAN, Hyderabad (IN); Eswaraiah SAJJA, Hyderabad (IN); Venkat Reddy GHOJALA, Hyderabad (IN); Venkataiah MALLAM, Hyderabad (IN)

(73) Assignee: MSN LABORATORIES PRIVATE LIMITED, R&D CENTER, Sangareddy (Dist), Hyderabad, Telangana (IN)

(21) Appl. No.: 17/789,460

(22) PCT Filed: Dec. 24, 2020
(86) PCT No.: PCT/IN2020/051049

§ 371 Date: Jun. 27, 2022

(30) Foreign Application Priority Data
Dec. 27, 2019 (IN) 201941054251

(51) Int. Cl. C07D 239/54

(52) U.S. Cl. CPC C07D 239/54

(57) ABSTRACT

The present invention relates to an improved process for the preparation of 4-({(1R)-2-[5-(2-fluoro-3-methoxy phenyl)-3-{[2-fluoro-6-(trifluoro methyl) phenyl] methyl}-4-methyl-2,6-dioxo-3,6-dihydropyrimidin-1(2H)-yl]-1-phenylethyl}amino) butanoic acid of formula (I) or its pharmaceutically acceptable salts. The compound of formula (I) is represented by the following structural formula:

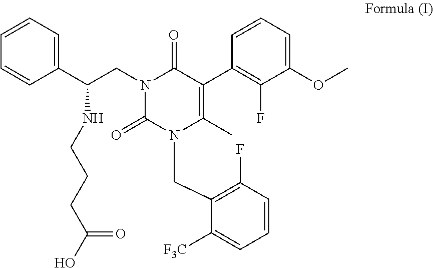 [SMILES: COc1cccc(-c2c(C)n(Cc3c(F)cccc3C(F)(F)F)c(=O)n(C[C@H](NCCCC(=O)O)c3ccccc3)c2=O)c1F]

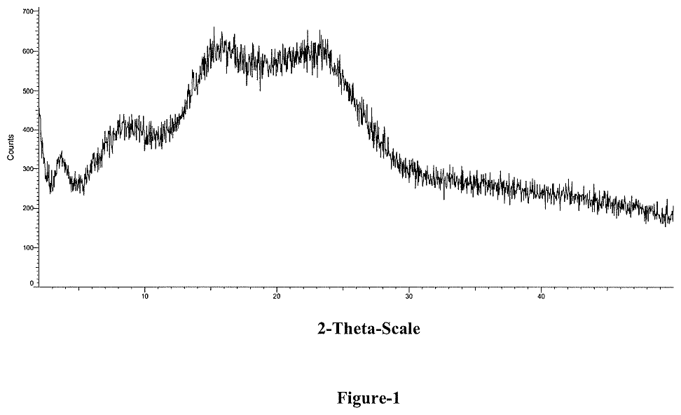

### RELATED APPLICATION

This patent application claims the benefit of priority of our Indian patent application number 201941054251 filed on 27 Dec. 2019 which is incorporated herein by reference.

### FIELD OF THE INVENTION

The present invention relates to an improved process for the preparation of 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoic acid of formula (I). The compound of formula (I) is represented by the following structural formula:

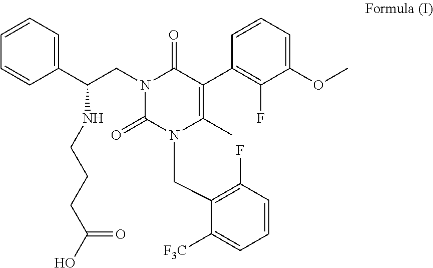 [SMILES: COc1cccc(-c2c(C)n(Cc3c(F)cccc3C(F)(F)F)c(=O)n(C[C@H](NCCCC(=O)O)c3ccccc3)c2=O)c1F]

### BACKGROUND OF THE INVENTION

4-({(IR)-2-[5-(2-Fluoro-3-methoxyphenyl)-3-{[2-fluoro-6-(trifluoromethyl) phenyl]methyl}-4-methyl-2,6-dioxo-3,6-dihydropyrimidin-1(2H)-yl]-1-phenylethyl}amino)butanoic acid is commonly known as “Elagolix”.

“Elagolix” is a gonadotropin-releasing hormone (GnRH) receptor antagonist indicated for the management of moderate to severe pain associated with endometriosis. USFDA-approved as “Elagolix sodium” with the brand name of ORILISSA® on Jul. 23, 2018 and it is available with dosage form of Eq 150 mg base, Eq 200 mg base tablet for oral administration.

U.S. Pat. No. 6,872,728 B2 discloses Elagolix and its pharmaceutically acceptable salts.

U.S. Pat. No. 7,056,927 B2 discloses a process for preparation of Elagolix, comprising reaction of N-[2-fluoro-6-(trifluoromethyl)benzyl]urea with diketene in presence of sodium iodide and trimethylsilyl chloride to get 1-[2-fluoro-6-(trifluoromethyl)benzyl]-6-methylpyrimidine-2,4(1H,3H)-dione of formula 1c which is further brominated to give 5-bromo-1-[2-fluoro-6-(trifluoromethyl)benzyl]-6-methylpyrimidine-2,4(1H,3H)-dione of formula 1d. The compound of formula 1d is coupled with N-t-Boc-D-phenylglycinol in presence of triphenylphosphine, tetrahydrofuran and di-tert-butyl azodicarboxylate to yield 5-bromo-1-[2-fluoro-6-(trifluoromethyl)benzyl]-6-methyl-3-[2(R)-tert-butoxycarbonylamino 2-phenylethyl]-pyrimidine-2,4(1H,3H)-dione of formula 1e which is further coupled with 2-fluoro-3-methoxyphenylboronic acid in presence sodium carbonate and tetrakis(triphenylphosphine) palladium water/dioxane in presence of, to get 3-[2(R)-amino-2-phenylethyl]-5-(2-fluoro-3-methoxyphenyl)-1-[2-fluoro-6-(trifluoromethyl)benzyl]-6-methyl-pyrimidine-2,4(1H,3H)-dione of formula if. The compound of formula if is coupled with ethyl 4-bromobutyrate in presence of diisopropylethylamine to get 3-[2(R)-{ethoxycarbonylpropyl-amino}-2-phenylethyl]-5-(2-fluoro-3-methoxy-phenyl)-1-[2-fluoro-6-(trifluoromethyl)benzyl]-6-methyl-pyrimidine-2,4(1H,-3H)-dione of formula Ig which is further hydrolyzed in presence of NaOH to get Elagolix sodium.

The main drawbacks of the above prior art process are as follows:
Low yield (about 26%) of Elagolix sodium prepared from 3-[2(R)-amino-2-phenylethyl]-5-(2-fluoro-3-methoxyphenyl)-1-[2-fluoro-6-(trifluoromethyl)benzyl]-6-methyl-pyrimidin e-2,4(1H,3H)-dione of formula 1f.
Forming triphenylphosphine oxide impurity due to use of triphenylphosphine which requires additional purifications.
Di-tert butyl azodicarboxylate exhibits hazardous and flammable properties. It is likely to explosions if catches with fire. Hence, its use is not safe in commercial scale preparations. Further, this reaction is also not viable for industrial scale-up.
Impurity-1 (desbromo impurity), Impurity-2 and Impurity-3 (dimer impurity) are formed during the preparation of compound of formula 1e

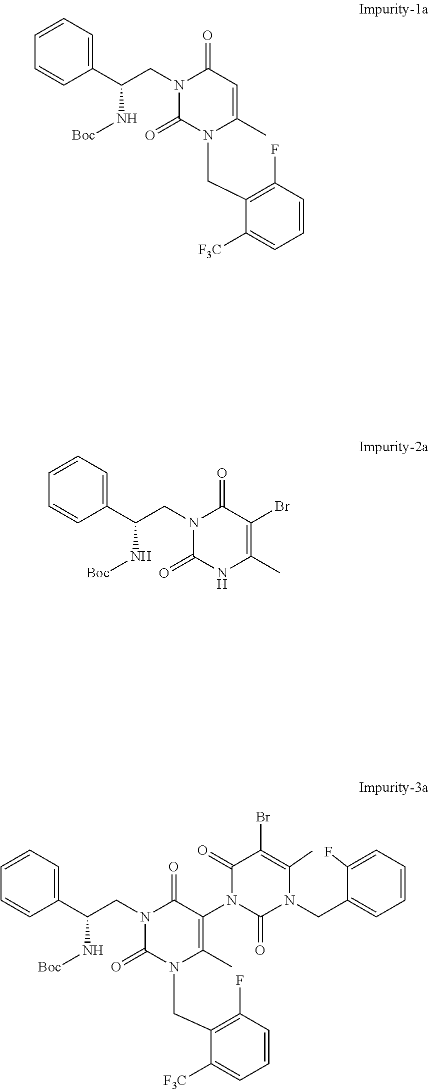 [SMILES: Cc1cc(=O)n(C[C@H](NC(=O)OC(C)(C)C)c2ccccc2)c(=O)n1Cc1c(F)cccc1C(F)(F)F ; Cc1[nH]c(=O)n(C[C@H](NC(=O)OC(C)(C)C)c2ccccc2)c(=O)c1Br ; Cc1c(-n2c(=O)c(Br)c(C)n(Cc3ccccc3F)c2=O)c(=O)n(C[C@H](NC(=O)OC(C)(C)C)c2ccccc2)c(=O)n1Cc1c(F)cccc1C(F)(F)F]

The above prior art also involves purification with silicagel (column purification) which is tedious, cumbersome and consumes large amount of solvents which leads to increase in the cost of production.

U.S. Pat. No. 8,765,948 B2 discloses a process for preparation of Elagolix sodium comprising reaction of (2-fluoro-6-trifluoromethyl-benzyl) urea with tert-butyl acetoacetate in presence of toluene to get N-((2-fluoro-6-(trifluoromethyl)benzyl)carbamoyl)-3-oxobutanamide which was cyclized in presence of p-toluenesulfonic acid monohydrate (PTSA) at reflux temperature to provide 1-(2-fluoro-6-trifluoromethyl-benzyl)-6-ethyl-1H-pyrimidine-2,4-dione of formula 1a which is further iodinated to provide 1-(2-fluoro-6-trifluoromethyl-benzyl)-5-iodo-6-methyl-1H-pyrimidine-2,4-dione of formula 1b. The compound of formula 1b is coupled with 2-fluoro-3-methoxyphenylboronic acid in presence of potassium hydroxide/water solution, tri-t-butyl phosphonium tetrafluoroborate and palladium acetate or 1,1-(bis-di-t-butylphosphino)ferrocene palladium dichloride to afford 5-(2-fluoro-3-methoxy-phenyl)-1-(2-fluoro-6-trifluoromethyl-benzyl)-6-methyl-1H-pyrimidine-2,4-dione of formula 1c which is further coupled with methanesulfonic acid (S)-3-tert-butoxycarbonylamino-3-phenyl-propyl ester in presence of dimethylformamide, potassium carbonate followed by work up with isopropyl acetate, methanesulfonic acid, potassium carbonate/water, 85% phosphoric acid/water to get 3-((R)-2-amino-2-phenyl-ethyl)-5-(2-fluoro-3-methoxy-phenyl)-1-(2-fluoro-6-trifluoromethyl-benzyl)-6-methyl-1H-pyrimidine-2,4-dione in isopropyl acetate of formula 1e which is further reacted with ethyl 4-bromobutyrate in presence of dimethylformamide and diisopropylethylamine followed by hydrolysis to get Elagolix.

Inventors of the present invention repeated the above prior art process as described in example 1 (step 1A) of U.S. Pat. No. 8,765,948 in scale-up level but suffering from degradation of N-((2-fluoro-6-(trifluoromethyl)benzyl)carbamoyl)-3-oxobutanamide (which formed before treatment with PTSA) to its starting material (i.e. (2-fluoro-6-trifluoromethyl-benzyl) urea) about 20% by HPLC due to cyclization carrying out in presence of PTSA monohydrate under reflux conditions.

Another drawback of above prior art process as described in example 1 (step-1A) of U.S. Pat. No. 8,765,948 and leads to formation of isomer impurity in N-((2-fluoro-6-(trifluoromethyl)benzyl)carbamoyl)-3-oxobutanamide about 8% by HPLC.

In view of the above, there is still a need to develop a commercially viable, inexpensive, simple, and eco-friendly process for the preparation of Elagolix. Inventors of the present invention have developed an improved process for the preparation of Elagolix which is simple and advantageous over the prior art processes.

### Advantages of the Present Invention

No degradation of N-((2-fluoro-6-(trifluoromethyl)benzyl)carbamoyl)-3-oxobutanamide of formula (II) to its starting material (1-(2-fluoro-6-(trifluoromethyl)benzyl)urea) during cyclization reaction in presence of acids like sulfuric acid and acetic acid or mixtures thereof in anhydrous conditions as these are not hydrates unlike p-toluenesulfonic acid monohydrate.
Dibromo impurity is arrested to very low level upon purifying the compound of formula (IV) by precipitation from a solvent or mixture of solvents.
Controlling of dimer impurity & des bromo impurity in the compound of formula (VI) by precipitation from a solvent or mixture of solvents thereof.
Substantial increase in the yield of Elagolix during the reaction of (R)-3-(2-amino-2-phenylethyl)-5-(2-fluoro-3-methoxyphenyl)-1-(2-fluoro-6-(trifluoromethyl)benzyl)-6-methylpyrimidine-2,4-(1H,3H)-dione with ethyl 4-bromo butanoate in the presence of phase transfer catalyst.

### SUMMARY OF THE INVENTION

In first, second, third, fourth, fifth embodiments, the present invention provides an improved process for the preparation of Elagolix of formula (I) or its pharmaceutically acceptable salts.

In sixth embodiment, the present invention provides a process for the preparation of compound of formula (III).

In seventh embodiment, the present invention provides pharmaceutical compositions comprising Elagolix sodium and one or more pharmaceutically acceptable carriers, excipients or diluents thereof.

### BRIEF DESCRIPTION OF THE DRAWINGS

FIG.-1: Illustrates Powdered X-Ray Diffraction (PXRD) pattern of amorphous form of Elagolix sodium.

### DETAILED DESCRIPTION OF THE INVENTION

The present invention relates to an improved process for the preparation of 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoic acid of formula (I) or its pharmaceutically acceptable salts.

The term “solvent” used in the present invention refers to “hydrocarbon solvents” such as n-hexane, n-heptane, cyclohexane, benzene, toluene, pentane, cycloheptane, ethyl benzene, m-, o-, or p-xylene or and the like; “ether solvents” such as dimethoxymethane, tetrahydrofuran, 1,3-dioxane, 1,4-dioxane, furan, diethyl ether, ethylene glycol dimethyl ether, ethylene glycol diethyl ether, diethylene glycol dimethyl ether, diethylene glycol diethyl ether, triethylene glycol dimethyl ether, anisole, methyl tert-butyl ether, 1,2-dimethoxy ethane and the like; “ester solvents” such as methyl acetate, ethyl acetate, isopropyl acetate, n-butyl acetate and the like; “polar-aprotic solvents” such as dimethylacetamide (DMA), dimethylformamide (DMF), dimethylsulfoxide (DMSO), N-methylpyrrolidone (NMP) and the like; “chloro solvents” such as methylene chloride, dichloroethane, chloroform, carbon tetrachloride, chlorobenzene and the like; “ketone solvents” such as acetone, methyl ethyl ketone, pentanone, methyl isobutylketone and the like; “nitrile solvents” such as acetonitrile, propionitrile, isobutyronitrile and the like; “alcohol solvents” such as methanol, ethanol, n-propanol, isopropanol, n-butanol, isobutanol, t-butanol, n-pentanol, isopentanol, 2-nitroethanol, ethylene glycol, 2-methoxyethanol, 1, 2-ethoxyethanol, diethylene glycol, 1, 2, or 3-pentanol, neo-pentyl alcohol, t-pentyl alcohol, diethylene glycol monoethyl ether, benzyl alcohol, phenol, or glycerol and the like; “polar solvents” such as water or mixtures thereof.

The term “base” used herein the present invention until unless specified is selected from inorganic bases like “alkali metal hydroxides” such as lithium hydroxide, sodium hydroxide, potassium hydroxide and the like; “alkali metal carbonates” such as sodium carbonate, potassium carbonate, lithium carbonate and the like; “alkali metal bicarbonates” such as sodium bicarbonate, potassium bicarbonate, lithium bicarbonate and the like; “alkali metal hydrides” such as sodium hydride, potassium hydride, lithium hydride and the like; ammonia; and organic bases such as triethyl amine, methyl amine, ethyl amine, diisopropylethylamine; “alkali metal alkoxides” such as sodium methoxide, sodium ethoxide, sodium tert-butoxide, potassium methoxide, potassium ethoxide, potassium tert-butoxide and the like or mixtures.

The term “protecting group” is selected from but not limited to trialkyl silyl such as trimethylsilyl (TMS), triethylsilyl (TES), triisopropylsilyl (TIPS), tert-butyldimethylsilyl (TBDMS), tertbutyldiphenylsilyl (TBDPS) and the like; acetyl group, benzyl group, benzoyl group, benzyloxycarbonyl group, trifluoroacetyl group, tert-butyl acetyl group, allyl group, methoxymethyl group, ethoxyethyl group, methoxyethoxymethyl group, p-methoxybenzyl halides, methylthiomethyl group, trityl group, benzyloxymethyl group, tert.butoxy carbonyl group (Boc), alkyl/arylsulfonic groups such as methanesulfonyl group (Ms), ethanesulfonyl group, benzenesulfonyl group, toluenesulfonyl group and the like.

The term “deprotection” is removal of a protecting group and it is carried out with “deprotecting agent”. Deprotecting agent can be selected based on the protecting group employed. The suitable deprotecting agent can be selected from but not limited to acids such as hydrochloric acid, hydrobromic acid, phosphoric acid, acetic acid, formic acid, trifluoroacetic acid, methane sulfonic acid, p-toluene sulfonic acid, camphor sulfonic acid and the like, bases such as alkali metal hydroxides, alkali metal carbonates, cesium 5 carbonate/imidazole, alkali metal bicarbonates, ammonia, cerium ammonium nitrate (CAN), 1,8-diazabicyclo[5.4.0]undec-7-ene (DBU), hydrogenating agents such as Pd, Pd/C, Pd(OH)2/C (Pearlman's catalyst), palladium acetate, platinum oxide (PtO₂), platinum black, sodium borohydride, BF₃-etherate, Raney-Ni, triethylsilane, trimethylsilyl halides, copper(II) chloride dihydrate and the like; fluoride ion sources such as potassium fluoride (KF), tetra butyl ammonium fluoride (TBAF), HF-pyridine, ammonium fluoride; trifluoromethane sulfonic acid (triflic acid), tris(dimethylamino)sulfonium difluorotrimethylsilicate (TASF), 2,3-dichloro-5,6-dicyano-1,4-benzoquinone (DDQ), acetyl chloride/methanol, N-iodosuccinimide in methanol and the like.

The term “phase transfer catalyst” is a quaternary ammonium salts such as tetra butyl ammonium bromide, tetrapropyl ammonium bromide, tributyl benzyl ammonium bromide, tetraoctyl ammonium bromide, tetra butyl ammonium iodide, tetra butyl ammonium hydrogen sulfate, benzyl trimethyl ammonium chloride, benzyl triethyl ammonium chloride, tetra butyl ammonium acetate, tetra butyl ammonium iodide, ethyl triphenyl phosphonium bromide, preferably tetra butyl ammonium bromide.

As used herein, the term “substantially free” refers to a compound of the present invention having one or more impurities less than about 2% or less than about 1% or 0.5% or less than about 0.4% or less than about 0.3% or less than about 0.2% or less than about 0.1% or less than about 0.05% or not detected.

In a first embodiment, the present invention provides an improved process for the preparation of Elagolix of formula (I) or pharmaceutically acceptable salts, comprises:
a) cyclizing the compound of formula (II) in presence of an acid under anhydrous conditions to produce the compound of formula (III),

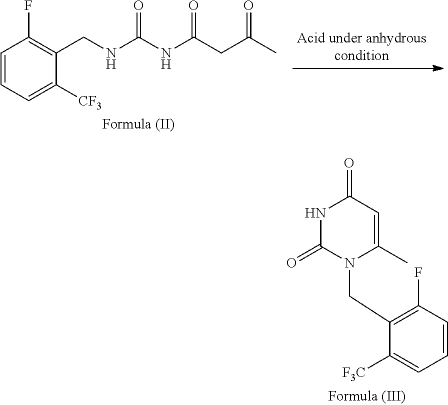 [SMILES: CC(=O)CC(=O)NC(=O)NCc1c(F)cccc1C(F)(F)F ; Cc1cc(=O)[nH]c(=O)n1Cc1c(F)cccc1C(F)(F)F]

b) converting the compound of formula (III) to Elagolix or its pharmaceutically acceptable salts.

In first aspect of first embodiment, wherein the acid is selected from sulfuric acid (H₂SO₄), acetic acid, polyphosphoric acid (H₃PO₄), nitric acid (HNO₃), hydrochloric acid (HCl), hydrobromic acid (HBr) or mixture of acids thereof.

In a second aspect of first embodiment, the obtained compound of formula (III) is substantially free from (1-(2-fluoro-6-(trifluoromethyl)benzyl)urea).

In a third aspect of first embodiment, the obtained compound of formula (III) is substantially free from isomer impurity of formula (IIIa)

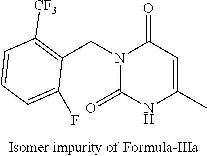 [SMILES: Cc1cc(=O)n(Cc2c(F)cccc2C(F)(F)F)c(=O)[nH]1]

In a fourth aspect of first embodiment, the present invention provides an improved process for the preparation of Elagolix of formula (I) or pharmaceutically acceptable salts, comprises:
a) cyclizing the compound of formula (II) in presence of mixture of concentrated sulfuric acid and acetic acid, to produce the compound of formula (III),

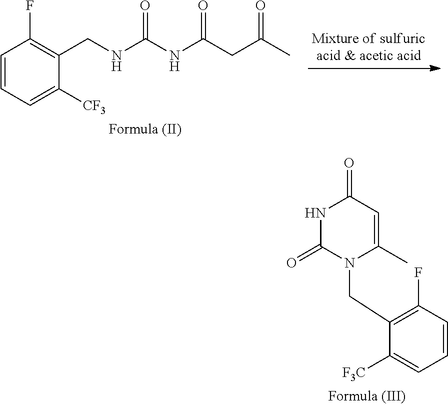 [SMILES: CC(=O)CC(=O)NC(=O)NCc1c(F)cccc1C(F)(F)F ; Cc1cc(=O)[nH]c(=O)n1Cc1c(F)cccc1C(F)(F)F]

b) converting the compound of formula (III) to Elagolix or its pharmaceutically acceptable salts.

In a second embodiment, the present invention provides an improved process for the preparation of Elagolix of formula (I) or pharmaceutically acceptable salts, comprises:
a) purifying 5-bromo-1-(2-fluoro-6-(trifluoromethyl)benzyl)-6-methylpyrimidine-2,4-(1H,3H)-dione of formula (IV)

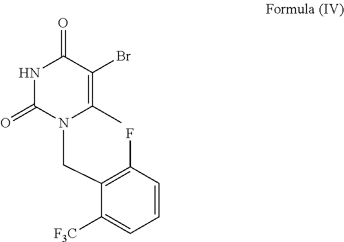 [SMILES: Cc1c(Br)c(=O)[nH]c(=O)n1Cc1c(F)cccc1C(F)(F)F]

to get the compound of formula (IV) substantially free from dibromo impurities of formulae-IVa and/or IVb

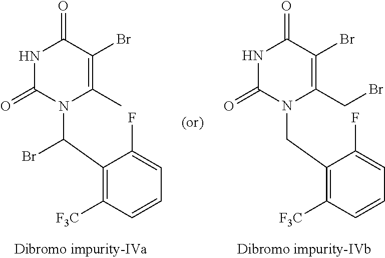 [SMILES: Cc1c(Br)c(=O)[nH]c(=O)n1C(Br)c1c(F)cccc1C(F)(F)F ; O=c1[nH]c(=O)n(Cc2c(F)cccc2C(F)(F)F)c(CBr)c1Br]

b) converting the pure compound of formula (IV) to Elagolix or its pharmaceutically acceptable salts.

In a first aspect of second embodiment, wherein purification is carried out by precipitation from a solvent or mixture of solvents thereof.

wherein the solvent is selected from polar aprotic solvents, ester solvents, nitrile solvents, alcohol solvents, ether solvent and the like and water. Prefarebly, mixture of polar aprotic solvents and water. More preferably, dimethylformamide and water.

In a third embodiment, the present invention provides an improved process for the preparation of Elagolix of formula (I) or pharmaceutically acceptable salts, comprises step-a) and/or step-b) of the following:
a) reacting the compound of formula (IV) with the compound of formula (V) in presence of a base, phase transfer catalyst in a solvent to provide the compound of formula (VI),

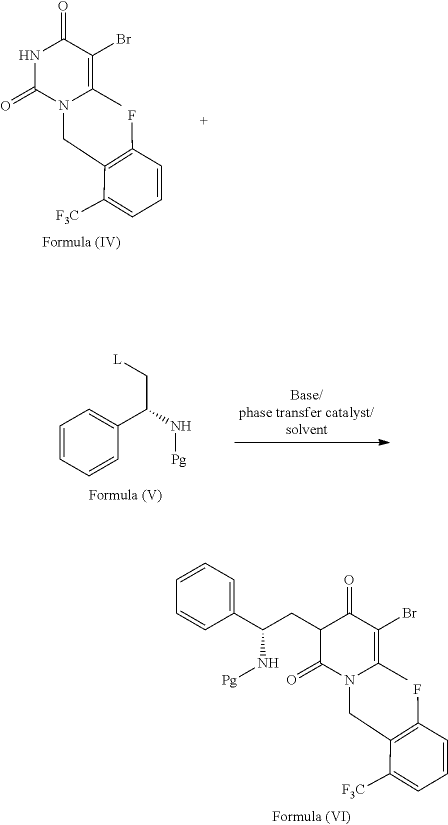

b) purifying the obtained compound of formula (VI) to get compound of formula (VI) which is substantially free from below impurities of formulae-1, 2 and 3

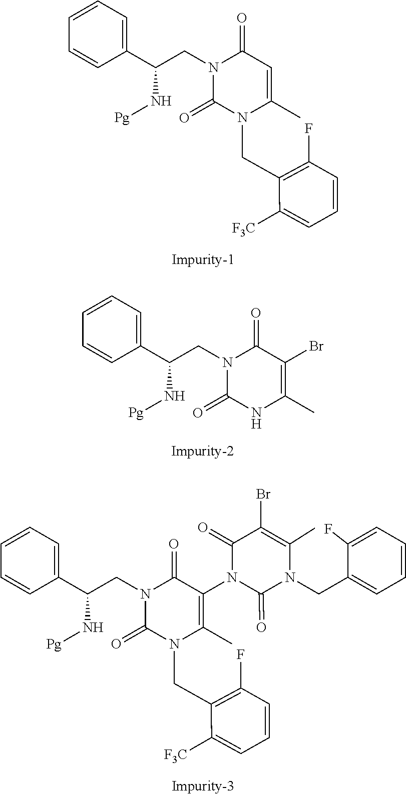

c) converting the compound of formula (VI) to Elagolix or its pharmaceutically acceptable salts.
   wherein L is a leaving group such as methanesulfonyloxy (—OMs), toluenesulfonyloxy (—OTs), chlorine, bromine, iodine and the like and Pg is a protecting group.

In first aspect of third embodiment, wherein the base used in step-a) is selected from alkali metal hydroxides such as lithium hydroxide, sodium hydroxide, potassium hydroxide and the like; alkali metal carbonates such as sodium carbonate, potassium carbonate, lithium carbonate and the like; alkali metal bicarbonates such as sodium bicarbonate, potassium bicarbonate, lithium bicarbonate and the like; the phase transfer catalyst used in step-a) is selected from quaternary ammonium salts such as tetra butyl ammonium bromide, tetra-butyl ammonium fluoride, tetrapropyl ammonium bromide, tributyl benzyl ammonium bromide, tetraoctyl ammonium bromide, tetra butyl ammonium iodide, tetra butyl ammonium hydrogen sulfate, benzyl trimethyl ammonium chloride, benzyl triethyl ammonium chloride, tetra butyl ammonium acetate, tetra butyl ammonium iodide, ethyl triphenyl phosphonium bromide, methyltributyl ammonium chloride, methyltrioctylammonium chloride, crown ethers. Preferably tetra butyl ammonium bromide; the solvent used in step-a) is selected from hydrocarbon solvents, alcohol solvents, polar aprotic solvent, ester solvents, nitrile solvents, ether solvents or mixtures thereof.

In a second aspect of third embodiment, wherein purification is carried out by a precipitation from a solvent or mixture of solvents selected from alcohol solvents, polar aprotic solvent, ester solvents, nitrile solvents, ether solvents thereof. Prefarebly, mixture of ethylene glycol and methanol.

In a third aspect of third embodiment, the present invention provides an improved process for the preparation of Elagolix of formula (I) or pharmaceutically acceptable salts, comprises step-a) and/or step-b) of the following:
a) reacting the compound of formula (IV) with the compound of formula (Va) in presence of a base, phase transfer catalyst in a solvent to provide the compound of formula (VIa),

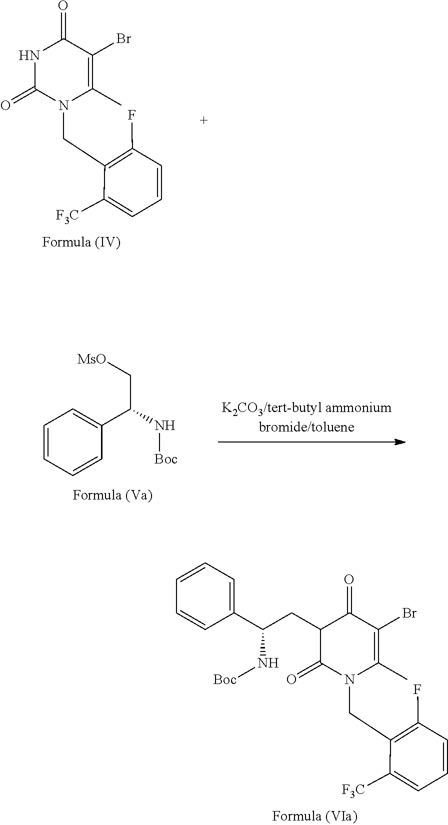 [SMILES: Cc1c(Br)c(=O)[nH]c(=O)n1Cc1c(F)cccc1C(F)(F)F ; CC(C)(C)OC(=O)N[C@@H](COS(C)(=O)=O)c1ccccc1 ; CC1=C(Br)C(=O)C(C[C@H](NC(=O)OC(C)(C)C)c2ccccc2)C(=O)N1Cc1c(F)cccc1C(F)(F)F]

b) purifying the obtained compound of formula (VIa) to get compound of formula (VIa) which is substantially free from below impurities of formulae-1a, 2a and 3a

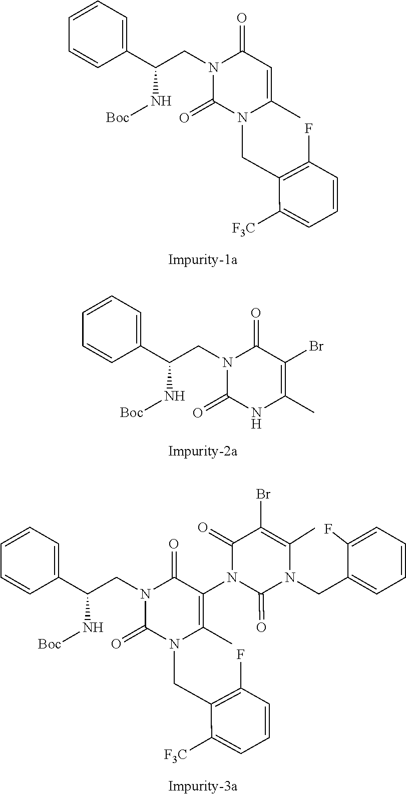 [SMILES: Cc1cc(=O)n(C[C@H](NC(=O)OC(C)(C)C)c2ccccc2)c(=O)n1Cc1c(F)cccc1C(F)(F)F ; Cc1[nH]c(=O)n(C[C@H](NC(=O)OC(C)(C)C)c2ccccc2)c(=O)c1Br ; Cc1c(-n2c(=O)c(Br)c(C)n(Cc3ccccc3F)c2=O)c(=O)n(C[C@H](NC(=O)OC(C)(C)C)c2ccccc2)c(=O)n1Cc1c(F)cccc1C(F)(F)F]

c) converting the compound of formula (VIa) to Elagolix or its pharmaceutically acceptable salts.

In a fourth embodiment, the present invention provides an improved process for the preparation of Elagolix of formula (I) or pharmaceutically acceptable salts, comprises:
a) reacting the compound of formula (VI) with 2-flouro3-methoxyphenylboronic acid of formula (VII) in a hydrocarbon solvent to provide the compound of formula (VIII) which is deprotected to produce the compound of formula (IX)

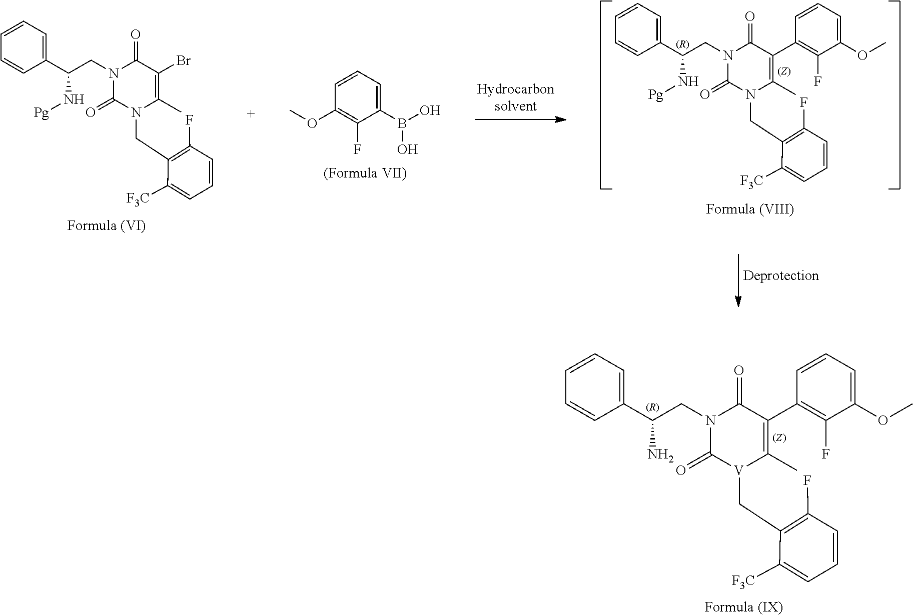

b) converting the compound of formula (IX) to Elagolix or its pharmaceutically acceptable salts.

In first aspect of fourth embodiment, wherein the hydrocarbon solvent is selected from toluene, n-hexane, n-heptane, cyclohexane, benzene, pentane, cycloheptane, m-, o-, or p-xylene or mixtures thereof in combination with other solvent selected from ether solvent, nitrile solvent, ester solvent, alcohol solvent and water or mixtures thereof. Preferably, toluene, 1,3-dioxane and water.

In second aspect of fourth embodiment, wherein the obtained compound of formula (IX) is substantially free from impurity-1b, impurity-2b and impurity-3b

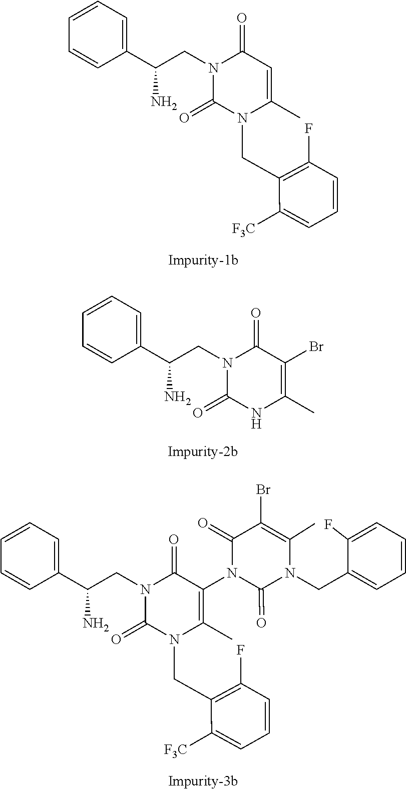 [SMILES: Cc1cc(=O)n(C[C@H](N)c2ccccc2)c(=O)n1Cc1c(F)cccc1C(F)(F)F ; Cc1[nH]c(=O)n(C[C@H](N)c2ccccc2)c(=O)c1Br ; Cc1c(-n2c(=O)c(Br)c(C)n(Cc3ccccc3F)c2=O)c(=O)n(C[C@H](N)c2ccccc2)c(=O)n1Cc1c(F)cccc1C(F)(F)F]

In a fourth embodiment, the present invention provides an improved process for the preparation of Elagolix of formula (I) or pharmaceutically acceptable salts, comprises:
a) reacting the compound of formula (VIa) with 2-flouro3-methoxyphenylboronic acid of formula (VII) in toluene to provide the compound of formula (VIIIa) which is deprotected to produce the compound of formula (IX)

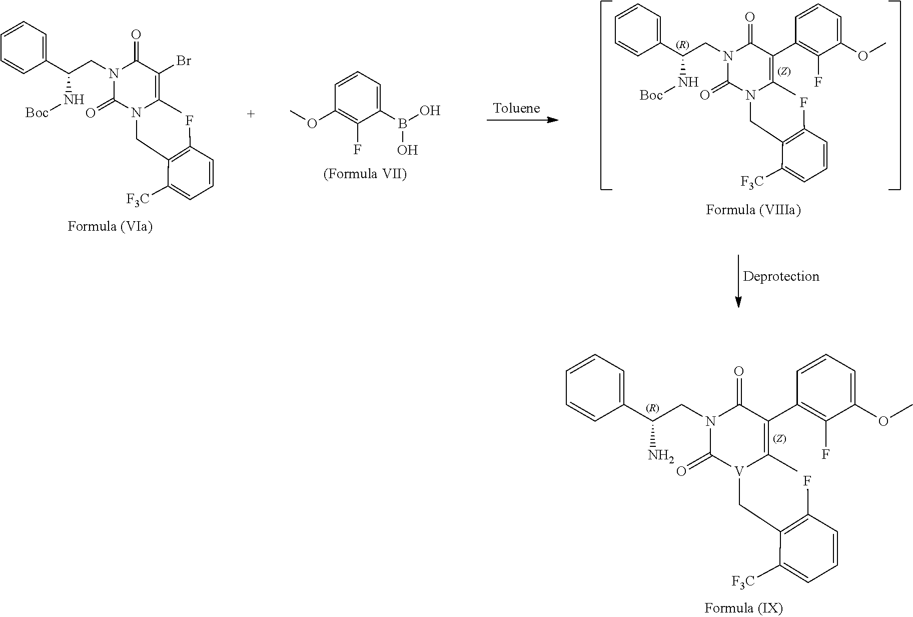

b) converting the pure compound of formula (IX) to Elagolix or its pharmaceutically acceptable salts.

In fifth embodiment, the present invention provides an improved process for the preparation of Elagolix of formula (I) or pharmaceutically acceptable salts, comprises:
a) reacting the compound of formula (IX) with the compound of formula (X) in presence of a base, phase transfer catalyst in a solvent to provide the compound of formula (XI)

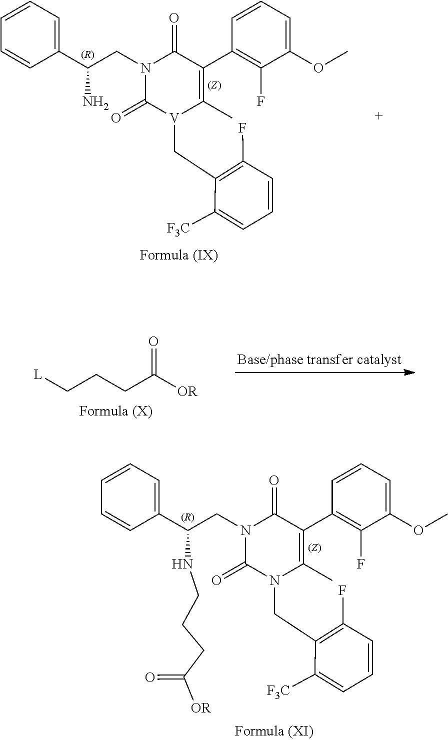

b) converting the compound of formula (XI) to get Elagolix or pharmaceutically acceptable salts
   wherein L is a leaving group such as chlorine, bromine, iodine, methanesulfonyloxy (—OMs), toluenesulfonyloxy (—OTs) and the like and R is selected from alkyl group having C₁-C₄ carbon atoms.

In first aspect of fifth embodiment, wherein the base used in step-a) is selected from potassium carbonate, sodium carbonate, sodium bicarbonate, potassium hydroxide, potassium carbonate, triethylamine, diisopropylethylamine, ammonia and the like; phase transfer catalyst is selected from quaternary ammonium salts like tetra-butylammonium bromide, tetra-butyl ammonium fluoride, benzyltriethyl ammonium chloride, methyltricapryl ammonium chloride, methyltributyl ammonium chloride, and methyltrioctylammonium chloride, crown ethers, and phosphonium compounds; solvent is selected from hydrocarbon solvents, alcohol solvents, ester solvents, nitrile solvents, chloro solvents, ether solvents or water or mixtures thereof; the conversion in step-b) is carried out by hydrolysis using a base or acid which is described hereinbefore.

In second aspect of fifth embodiment, the present invention provides an improved process for the preparation of Elagolix of formula (I) or pharmaceutically acceptable salts, comprises:
a) reacting the compound of formula (IX) with the compound of formula (X) in presence of a base, phase transfer catalyst in a solvent to provide the compound of formula (XIa)

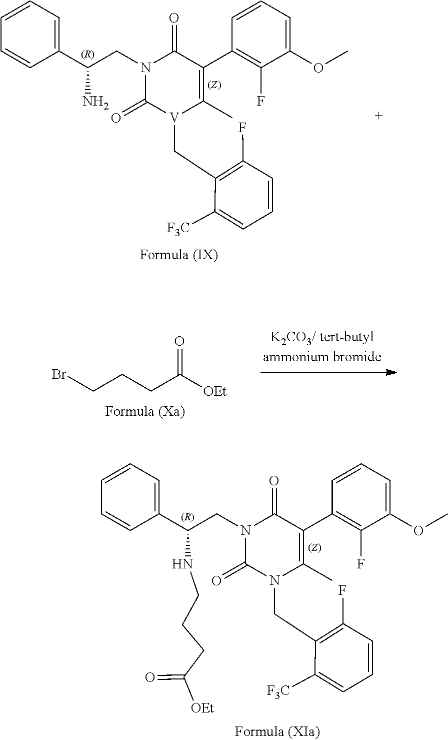

b) converting the compound of formula (XIa) to get Elagolix or pharmaceutically acceptable salts

In sixth embodiment, the present invention provides a process for the preparation of compound of formula (III), comprising reacting of compound of formula (X) with compound of formula (XI) in presence of base in a solvent to provide the compound of formula (III)

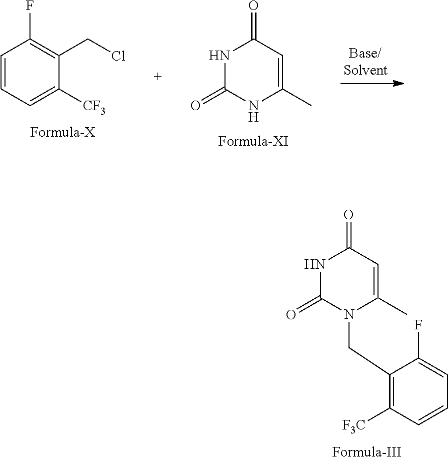

wherein the base and solvent are same as defined hereinbefore.

In seventh embodiment, the present invention provides pharmaceutical compositions comprising a therapeutically effective amount of sodium 4-({(1R)-2-[5-(2-fluoro-3-methoxyphenyl)-3-{[2-fluoro-6-(trifluoromethyl)phenyl]methyl}-4-methyl-2,6-dioxo-3,6-di hydropyrimidin-1(2H)-yl]-1-phenylethyl}amino)butanoate and one or more pharmaceutically acceptable carriers, excipients or diluents

wherein pharmaceutical compositions containing pure sodium 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate of the present invention may be prepared by using diluents or excipients such as fillers, bulking agents, binders, wetting agents, disintegrating agents, surface active agents, and lubricants. Various modes of administration of the pharmaceutical compositions of the invention can be selected depending on the therapeutic purpose, for example tablets, pills, powders, liquids, suspensions, emulsions, granules, capsules, suppositories, or injection preparations.

The compounds of formulae III, IV, VI, IX and XI obtained by the present invention can be converted to the Elagolix as described in the examples or any other process known from the art.

The HPLC analysis of Elagolix sodium prepared by the present invention was analyzed by HPLC under the following conditions:

Apparatus: A liquid chromatographic system equipped with variable wavelength UV detector; Column: YMC trait C18, 250*4.6 mm, 5 mm (or) equivalent; Column temperature: 50° C.; Wave length: 225 nm; Injection volume: 5 μl; Diluent: acetonitrile: water (80:20) % v/v; Buffer: transfer 2.7 gms of potassium dihydrogen phosphate in 1000 mL of Milli-Q-water. Filter this solution through filter paper; Mobile phase-A: Buffer (100%) Mobile phase-B: Methanol: Buffer: Acetonitrile (69:20:11)% v/v.

The process described in the present invention was demonstrated in examples illustrated below. These examples are provided as illustration only and therefore should not be construed as limitation of the scope of the invention:

### EXAMPLES

### Example-1: Preparation of 1-(2-fluoro-6-(trifluoromethyl)benzyl)-6-methylpyrimidine-2,4(1H,3H)-dione

Toluene (2000 ml) was added to 1-(2-fluoro-6-(trifluoromethyl)benzyl)urea (100 gm) at 25 to 30° C. Tert-butyl acetoacetate (470 gm) was added to above mixture and heated the reaction mixture to 110-115° C. and stirred for 10 hrs at same temperature. Cooled the mixture to 65-70° C. and distilled off the solvent from the mixture. n-Heptane was added to the obtained residue and stirred for 2 hrs. Filtered the obtained solid and washed with n-heptane to get N-((2-fluoro-6-(trifluoromethyl)benzyl)carbamoyl)-3-oxobutanamide. Acetic acid and sulfuric acid were added to the obtained compound at 65-70° C. and stirred for 2 hrs at same temperature. Cooled the reaction mixture to 15-20° C. and added water and stirred for 2 hrs at same temperature. Filtered the precipitated solid and added ethyl acetate and isopropanol and stirred for 2 hrs. Filtered the solid, washed with ethyl acetate and then dried to get the title compound.

(Yield: 66 gms, Purity: 99.04% by HPLC, N-((2-fluoro-6-(trifluoromethyl)benzyl) carbamoyl)-3-oxobutanamide impurity: 0.01%, isomer impurity: Not detected, M.P: 207-209° C.)

### Example-2: Preparation of 5-bromo-1-(2-fluoro-6-(trifluoromethyl)benzyl)-6-methylpyrimidine 2,4(1H,3H)-dione

Acetic acid (300 ml) was added to 1-(2-fluoro-6-(trifluoromethyl)benzyl)-6-methylpyrimidine 2,4(1H,3H)-dione (100 gm) at 25-30° C. Bromine (51.23 ml) was added to the above reaction mixture at 10-15° C. and stirred for about 2 hrs at 25-30° C. Aqueous sodium metabisulphate solution was added to the obtained reaction mixture and stirred for 30 min. Filtered the solid and washed with water. Dimethylformamide and water were added to the obtained wet compound and stirred for about 30 mins. Filtered the obtained solid, washed with water and then dried to get title compound.

(Yield: 100 gms, purity: 99.36% by HPLC)

### Example-3: Preparation of 5-bromo-1-(2-fluoro-6-(trifluoromethyl)benzyl)-6-methylpyrimidine 2,4(1H,3H)-dione

2,2-Dimethyl-1,3-dioxane-4,6-dione (201.3 gms) was added to methylene chloride (1600 ml), pyridine (328.2 gms) at 25-30° C. Cooled the reaction mixture to −10° C. to −5° C. and added acetyl chloride (199.4 gms) and stirred for 2 hrs at same temperature. Raised the temperature of the reaction mixture to 15-5° C. and washed with hydrochloride solution. Separated the layers and washed the organic layer with aqueous sodium chloride solution. Separated the layers and distilled off the solvent from the organic layer. Dissolved the obtained residue in methyl tert-butyl ether and filtered through hyflow bed. Distilled off the solvent completely from the filtrate and slurried the obtained compound in n-heptane followed by water. Filtered the obtained compound and then dried to get 5-(1-hydroxyethylidene)-2,2-dimethyl-1,3-dioxane-4,6-dione.

Above obtained 5-(1-hydroxyethylidene)-2,2-dimethyl-1,3-dioxane-4,6-dione and 1-(2-fluoro-6-(trifluoromethyl)benzyl)urea (100 gms) were added to toluene (1000 ml). Heated the reaction mixture to 110° C. and stirred for 2 hrs at same temperature to get N-((2-fluoro-6-(trifluoromethyl)benzyl)carbamoyl)-3-oxobutanamide. Sulfuric acid was added to the above reaction mixture and stirred for 2 hrs. Distilled off the solvent from the mixture followed by co-distillation with isopropanol. Isopropanol was added to the obtained residue at 25-30° C. and stirred for 90 min at same temperature. Filtered the solid and washed with isopropanol. Mixture of ethyl acetate and isopropanol was added to the obtained solid at 50-55° C. and stirred for 90 min. Cooled the obtained compound to 40-45° C. and stirred for 30 min. Filtered the solid, washed with ethyl acetate and then dried to get the title compound (Yield: 80 gms, 1-(2-fluoro-6-(trifluoromethyl)benzyl)urea: 0.02%, isomer impurity of formula-IIIa: 0.07% & N-((2-fluoro-6-(trifluoromethyl)benzyl)carbamoyl)-3-oxobutanamide: not detected)

### Example-4: Preparation of (R)-2-((tert-butoxycarbonyl)amino-2-phenylethyl methanesulfonate

Dimethylfornamide (280 ml), triethylamine (116.7 gm) were added to (R)-tert-butyl (2-hydroxy-1-phenylethyl)carbamate (137 gm). Methanesulfonyl chloride (100 gm) was added to the above reaction mixture at 0-5° C. Raised the temperature of the reaction mixture to 25-30° C. and stirred for about 4 hrs at same temperature. Acetone and water were added to the obtained reaction mixture at 0-5° C. and stirred for 1 hr. Filtered the solid, washed with acetone and water mixture and then dried to provide (R)-2-((tert-butoxycarbonyl)amino-2-phenylethyl methanesulfonate.

### Example-5: Preparation of (R)-tert-butyl (2-(5-bromo-3-(2-fluoro-6-(trifluoromethyl) benzyl)-4-methyl-2,6-dioxo-2,3-dihydropyrimidin-1(6H)-yl)-1-phenylethyl)carbamate

Toluene (600 ml), water (300.0 ml) were added to 5-bromo-1-(2-fluoro-6-(trifluoromethyl)benzyl)-6-methylpyrimidine-2,4(1H,3H)-dione (100 gms) at 25-30° C. Potassium carbonate (92 gms), tetrabutylammonium bromide (42 gms) and the compound of example-4 were added to the above reaction mixture at 25-30° C. Heated the reaction mixture to 55-60° C. and stirred for 4 hrs at same temperature. Separated the organic layer and washed the organic layer with water. Distilled off the solvent from the organic layer. Dissolved the obtained crude in ethyl acetate and added to pre-cooled n-heptane, stirred for about 2 hrs. Filtered the precipitated solid and added to ethyl acetate and methanol followed by heating the reaction mixture to 55-60° C. and stirred for about 1 hr at same temperature. Cooled the reaction mixture to 25-30° C. and stirred for about 1 hr at same temperature. Filtered the obtained solid, washed with the mixture of ethylene glycol and methanol followed by water and then dried to get the title compound.

(Yield: 90 gms, purity: 99.29% by HPLC, Impurity-1a: 0.28%, Impurity-2a: 0.20%, Impurity-3a: Not detected).

### Example-6: Preparation of (R)-3-(2-amino-2-phenylethyl)-5-(2-fluoro-3-methoxyphenyl)-1-(2-fluoro-6-(trifluoromethyl)benzyl)-6-methylpyrimidin-2,4-(1H,3H)-dione

Dioxane (125 ml), water (125 ml), toluene (250 ml), Na₂CO₃ (53.0 gm) were added to (R)-tert-butyl (2-(5-bromo-3-(2-fluoro-6-(trifluoromethyl)benzyl)-4-methyl-2,6-dioxo-2,3-dihydropyrimidin-1(6H)-yl)-1-phenylethyl)carbamate (50 gins) and (2-fluoro-3-methoxyphenyl)boronic acid (21.11 gm) at 25-30° C. and stirred for about 2 hrs under nitrogen atmosphere. Tetrakistriphenylphosphine palladium (0) (9.0 gm) was added to the above reaction mixture at 90-95° C. and stirred for 4 hrs. Methyl tert-butyl ether and water were added to the obtained reaction mixture followed by charcoal and stirred for 30 min. Filtered the reaction mixture through hy-flow bed and separated the organic and aqueous layers from filtrate. Extracted the aqueous layer with methyl tert butyl ether. Combined the total organic layers and distilled off the solvent under reduced pressure. Dissolved the obtained residue in tetrahydrofuran (500 ml) and added con. hydrochloric acid (50 ml) to it and stirred for about 2 hrs at 50-55° C.

Distilled off the solvent from the reaction mixture under reduced pressure to get residue. Toluene, water and hydrochloride were added to the obtained residue and stirred for 30 min. Separated the organic layer and washed with aqueous sodium carbonate solution. Separated the layers and extracted the compound from organic layer with aqueous phosphoric acid solution. Separated the layers and washed the aqueous layer with isopropyl acetate. Separated the layers and basified the aqueous layer with aqueous sodium bicarbonate solution. Extracted the aqueous layer with ethyl acetate. Distilled off the solvent from the organic layer under reduced pressure to afford the title compound.

(Yield: 17 gr. Purity: 99.40% by HPLC, Impurity-1b: 0.06%, Impurity-2b: 0.09%)

### Example-7: Preparation of sodium (R)-4-((2-(5-(2-fluoro-3-methoxyphenyl)-3-(2-fluoro-6-(trifluoromethyl)benzyl)-4-methyl-2,6-dioxo-2,3-dihydropyrimidin-1(6H)-yl)-1-phenyl ethyl)amino)butanoate

(R)-3-(2-Amino-2-phenylethyl)-5-(2-fluoro-3-methoxyphenyl)-1-(2-fluoro-6-(trifluoro methyl)benzyl)-6-methylpyrimidine-2,4-(1H,3H)-dione (50 gm), dimethylacetamide (250 ml), ethyl 4-bromo butanoate (14.3 gms) and diisopropylethylamine (14.2 gms) were charged into a round bottom flask at 25-30° C. Heated the reaction mixture to 50-55° C. and stirred for 2 hrs. Water and isopropyl acetate were added to the obtained reaction mixture and stirred for 20 min. Separated the aqueous, organic layers and distilled off the solvent from the organic layer under reduced pressure. Ethanol and aqueous sodium hydroxide solution were added to the obtained residue and stirred for about 2 hrs at 40-45° C. Distilled off the solvent from the reaction mixture under reduced pressure. Water and isopropyl acetate were added to the obtained reaction mixture and stirred for 20 min. Separated the layers and extracted the aqueous layer with isopropyl acetate. Ethyl acetate and sodium chloride were added to the aqueous layer and stirred for 30 min. Separated the layers and extracted the aqueous layer with ethyl acetate. Combined the total organic layers and added charcoal for particle free solution. Filtered the solution through hy-flow bed and distilled off the solvent from the organic layer to provide the title compound.

(Yield: 35 gms, Purity: 99.77% by HPLC)

### Example-8: Preparation of (R)-ethyl 4-((2-(5-(2-fluoro-3-methoxyphenyl)-3-(2-fluoro-6-(trifluoromethyl)benzyl)-4-methyl-2,6-dioxo-2,3-dihydropyrimidin-1(6H)-yl)-1-phenylethyl)amino)butanoate

(R)-3-(2-Amino-2-phenylethyl)-5-(2-fluoro-3-methoxyphenyl)-1-(2-fluoro-6-(trifluoro methyl)benzyl)-6-methylpyrimidine-2,4-(1H,3H)-dione (100 gm), toluene (500 ml), ethyl 4-bromo butanoate (178 gms), sodium carbonate (23.3 gms), tert-butyl ammonium bromide (11.5 gms) were charged into a round bottom flask at 25-30° C. Heated the reaction mixture to 90-95° C. and stirred for 6 hrs. Toluene and water were added to the obtained reaction mixture and stirred for 20 min. Separated the aqueous, organic layers and extracted the product from organic layer with aqueous phosphoric acid solution. Separated the layers and methylene chloride was added to the aqueous layer. Basified the reaction mixture with aqueous sodium carbonate solution. Separated the layers and distilled off the solvent from organic layer to get the title compound. (Yield: 110 gms)

### Example-9: Preparation of sodium (R)-4-((2-(5-(2-fluoro-3-methoxyphenyl)-3-(2-fluoro-6-(trifluoromethyl)benzyl)-4-methyl-2,6-dioxo-2,3-dihydropyrimidin-1(6H)-yl)-1-phenyl ethyl)amino)butanoate

To the compound obtained from example-8 added isopropanol (500 ml) and aqueous sodium hydroxide solution (36.6 gms dissolved in 500 ml of water) at 25-30° C. and stirred for about 1 hr at same temperature. Distilled off the solvent from the reaction mixture under reduced pressure. Water, acetonitrile and methyl tert-butyl ether were added to the obtained reaction mixture and stirred for 20 min. Separated the layers and washed the aqueous layer with the mixture of acetonitrile and methyl tert-butyl ether. Separated the layers and added mixture of acetonitrile and methyl tert-butyl ether to the aqueous layer. Basified the mixture with aqueous sodium carbonate solution. Separated the layers and extracted the aqueous layer with acetonitrile and methyl tert-butyl ether. Combined the total organic layers and treated with charcoal for particle free solution. Filtered the solution through hyflow bed and washed the bed with methanol. Combined total organic layers and distilled off the solvent followed by co-distillation with methanol. Cyclohexane is added to the obtained compound and stirred for 30 min at 25-30° C. Filtered the obtained compound and then dried to get the title compound. (Yield: 80 gms, M.R: 102-104° C., purity: 99.83 by HPLC).

The PXRD pattern of the obtained compound is illustrated in FIGURE-1.

1. Improved process for the preparation of Elagolix of formula (I) or pharmaceutically acceptable salts, 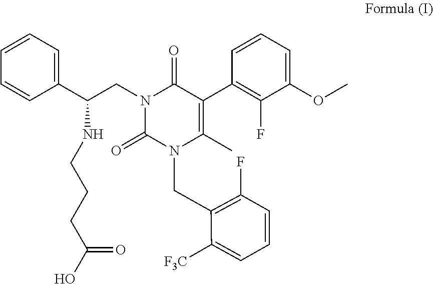 [SMILES: COc1cccc(-c2c(C)n(Cc3c(F)cccc3C(F)(F)F)c(=O)n(C[C@H](NCCCC(=O)O)c3ccccc3)c2=O)c1F]
comprising:
a) cyclizing the compound of formula (II) in presence of an acid under anhydrous conditions to produce the compound of formula (III), 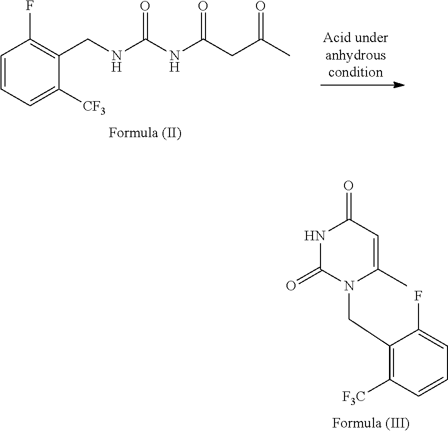 [SMILES: CC(=O)CC(=O)NC(=O)NCc1c(F)cccc1C(F)(F)F ; Cc1cc(=O)[nH]c(=O)n1Cc1c(F)cccc1C(F)(F)F]
b) converting the compound of formula (III) to Elagolix or its pharmaceutically acceptable salts.

2. The process of claim 1, wherein the acid is selected from sulfuric acid (H₂SO₄), acetic acid, polyphosphoric acid (H₃PO₄), nitric acid (HNO₃), hydrochloric acid (HCl), hydrobromic acid (HBr) or mixture of acids thereof.

3. The process of claim 1, the compound of formula (III) is substantially free from (1-(2-fluoro-6-(trifluoromethyl)benzyl)urea).

4. The process of claim 1, comprises,
a) cyclizing the compound of formula (II) in presence of mixture of sulfuric acid and acetic acid to produce the compound of formula (III), 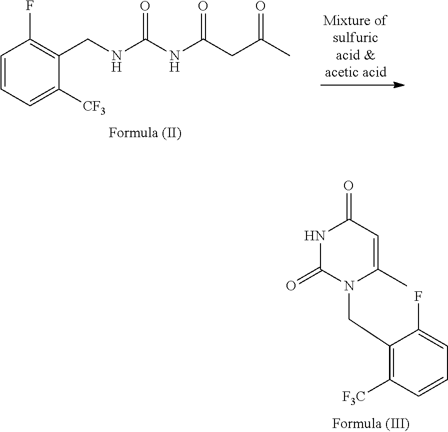 [SMILES: CC(=O)CC(=O)NC(=O)NCc1c(F)cccc1C(F)(F)F ; Cc1cc(=O)[nH]c(=O)n1Cc1c(F)cccc1C(F)(F)F]
b) converting the compound of formula (III) to Elagolix or its pharmaceutically acceptable salts.

5. Improved process for the preparation of Elagolix of formula (I) or pharmaceutically acceptable salts, 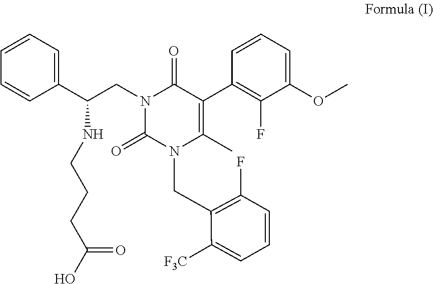 [SMILES: COc1cccc(-c2c(C)n(Cc3c(F)cccc3C(F)(F)F)c(=O)n(C[C@H](NCCCC(=O)O)c3ccccc3)c2=O)c1F]
comprising:
a) purifying 5-bromo-1-(2-fluoro-6-(trifluoromethyl)benzyl)-6-methylpyrimidine-2,4-(1H,3H)-dione of formula (IV) 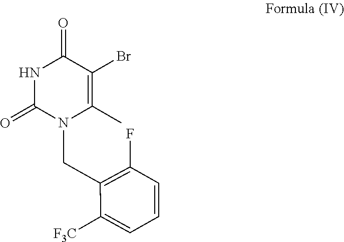 [SMILES: Cc1c(Br)c(=O)[nH]c(=O)n1Cc1c(F)cccc1C(F)(F)F]
b) converting the pure compound of formula (IV) to Elagolix or its pharmaceutically acceptable salts.

6. The process of claim 5, wherein the pure compound of formula (IV) is substantially free from impurities of formula-IVa or IVb

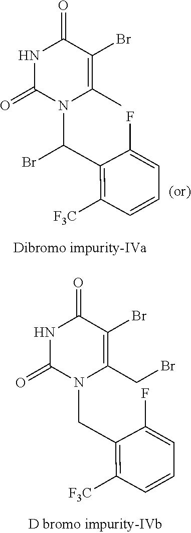 [SMILES: Cc1c(Br)c(=O)[nH]c(=O)n1C(Br)c1c(F)cccc1C(F)(F)F ; O=c1[nH]c(=O)n(Cc2c(F)cccc2C(F)(F)F)c(CBr)c1Br]

7. The process of claim 5, wherein purifying is carried out by precipitation from a solvent or mixture of solvents thereof.

8. The process claim 7, wherein the solvent is selected from polar aprotic solvent, ester solvents, nitrile solvents, alcohol solvents, ether solvent and the like and water,

9. Improved process for the preparation of Elagolix of formula (I) or pharmaceutically acceptable salts, 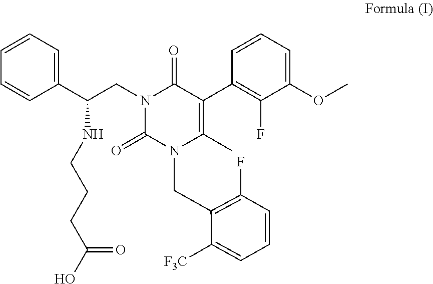 [SMILES: COc1cccc(-c2c(C)n(Cc3c(F)cccc3C(F)(F)F)c(=O)n(C[C@H](NCCCC(=O)O)c3ccccc3)c2=O)c1F]
comprises step-a) and/or step-b) of the following:
a) reacting the compound of formula (IV) with the compound of formula (V) in presence of a base, phase transfer catalyst in a solvent to provide the compound of formula (VI), 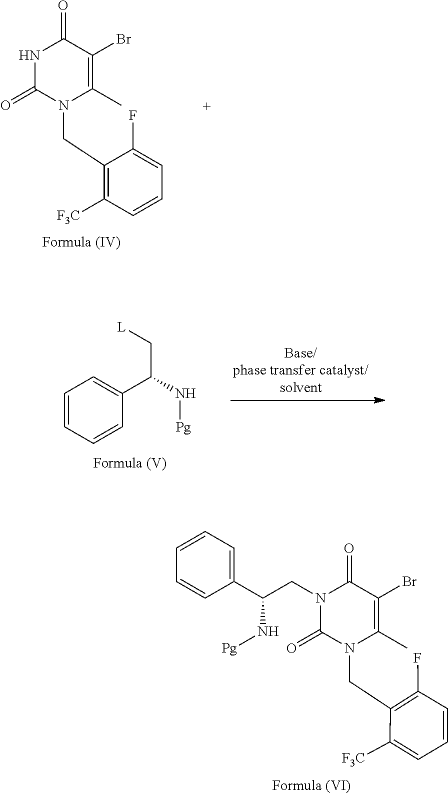
b) converting the compound of formula (VI) to Elagolix or its pharmaceutically acceptable salts.
wherein L is a leaving group and Pg is a protecting group.

10. The process of claim 9, wherein the compound of formula (VI) is substantially free from impurities of formulae-1, 2 and 3

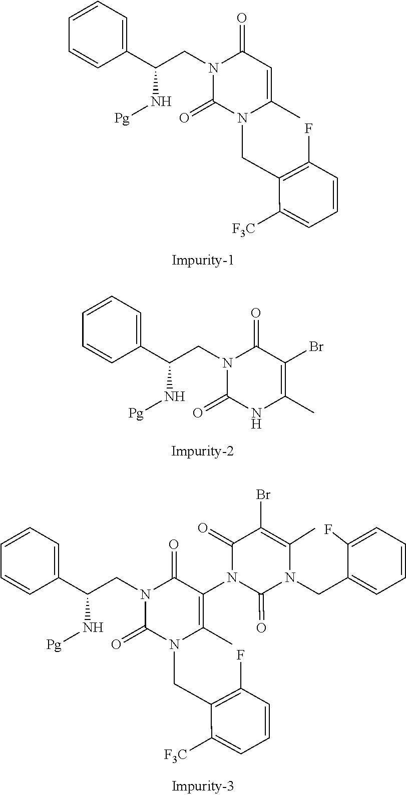

11. The process of claim 9, wherein the leaving group is selected from methanesulfonyloxy (—OMs), toluenesulfonyloxy (—OTs), chlorine, bromine, iodine and the like.

12. The process of claim 9, wherein the base used in step-a) is selected from alkali metal hydroxides such as lithium hydroxide, sodium hydroxide, potassium hydroxide and the like; alkali metal carbonates such as sodium carbonate, potassium carbonate, lithium carbonate and the like; alkali metal bicarbonates such as sodium bicarbonate, potassium bicarbonate, lithium bicarbonate and the like; the phase transfer catalyst used in step-a) is selected from quaternary ammonium salts such as tetra butyl ammonium bromide, tetra-butyl ammonium fluoride, tetrapropyl ammonium bromide, tributyl benzyl ammonium bromide, tetraoctyl ammonium bromide, tetra butyl ammonium iodide, tetra butyl ammonium hydrogen sulfate, benzyl trimethyl ammonium chloride, benzyl triethyl ammonium chloride, tetra butyl ammonium acetate, tetra butyl ammonium iodide, ethyl triphenyl phosphonium bromide, methyltributyl ammonium chloride, methyltrioctylammonium chloride, crown ethers.

13. The process of claim 9, wherein the phase transfer catalyst is tetra butyl ammonium bromide; the solvent used in step-a) is selected from hydrocarbon solvents, alcohol solvents, polar aprotic solvent, ester solvents, nitrile solvents, ether solvents or mixtures thereof.

14. Improved process for the preparation of Elagolix of formula (I) or pharmaceutically acceptable salts, 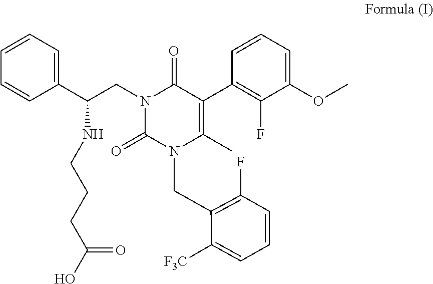 [SMILES: COc1cccc(-c2c(C)n(Cc3c(F)cccc3C(F)(F)F)c(=O)n(C[C@H](NCCCC(=O)O)c3ccccc3)c2=O)c1F]
comprises step-a) and/or step-b) of the following:
a) reacting the compound of formula (IV) with the compound of formula (Va) in presence of a base, phase transfer catalyst in a solvent to provide the compound of formula (VIa), 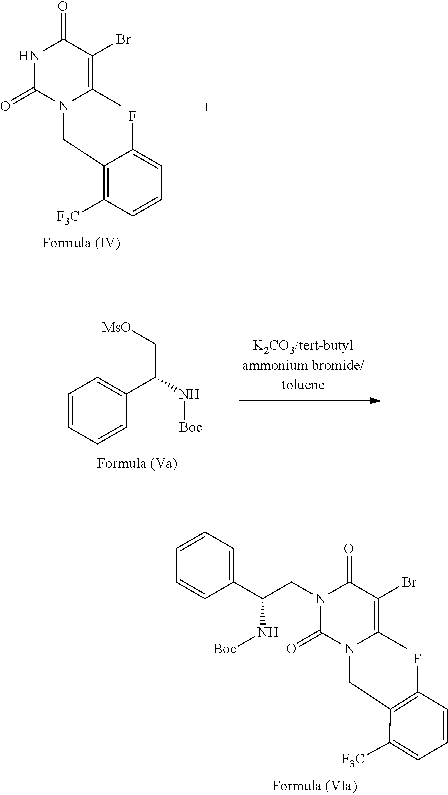 [SMILES: Cc1c(Br)c(=O)[nH]c(=O)n1Cc1c(F)cccc1C(F)(F)F ; CC(C)(C)OC(=O)N[C@@H](COS(C)(=O)=O)c1ccccc1 ; Cc1c(Br)c(=O)n(C[C@H](NC(=O)OC(C)(C)C)c2ccccc2)c(=O)n1Cc1c(F)cccc1C(F)(F)F]
b) converting the compound of formula (VIa) to Elagolix or its pharmaceutically acceptable salts.

15. Improved process for the preparation of Elagolix of formula (I) or pharmaceutically acceptable salts, 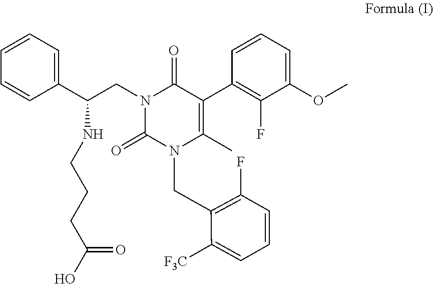 [SMILES: COc1cccc(-c2c(C)n(Cc3c(F)cccc3C(F)(F)F)c(=O)n(C[C@H](NCCCC(=O)O)c3ccccc3)c2=O)c1F]
comprises:
a) reacting the compound of formula (VI) with 2-flouro3-methoxyphenylboronic acid of formula (VII) in a hydrocarbon solvent to provide the compound of formula (VIII) which is deprotected to produce the compound of formula (IX) 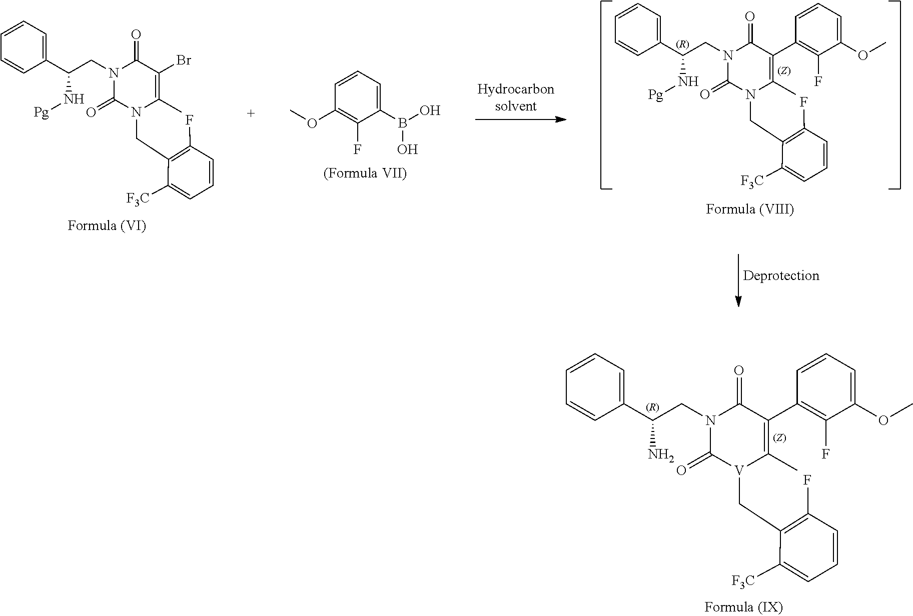
b) converting the pure compound of formula (IX) to Elagolix or its pharmaceutically acceptable salts.

16. The process of claim 15, wherein the hydrocarbon solvent is selected from toluene, n-hexane, n-heptane, cyclohexane, benzene, pentane, cycloheptane, in-, o-, or p-xylene or mixtures thereof in combination with other solvent selected from ether solvent, nitrile solvent, ester solvent, alcohol solvent and water or mixtures thereof; preferably, toluene, 1,3-dioxane and water.

17. The process of claim 15, wherein the obtained compound of formula (IX) is substantially free from impurity-1b, impurity-2b and impurity-3b

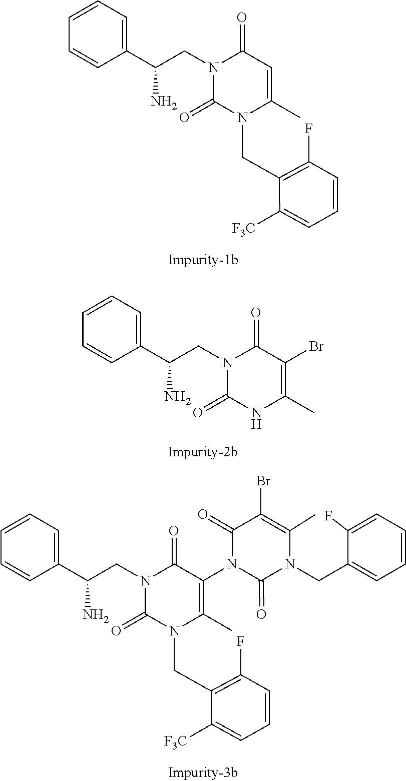 [SMILES: Cc1cc(=O)n(C[C@H](N)c2ccccc2)c(=O)n1Cc1c(F)cccc1C(F)(F)F ; Cc1[nH]c(=O)n(C[C@H](N)c2ccccc2)c(=O)c1Br ; Cc1c(-n2c(=O)c(Br)c(C)n(Cc3ccccc3F)c2=O)c(=O)n(C[C@H](N)c2ccccc2)c(=O)n1Cc1c(F)cccc1C(F)(F)F]

18. Improved process for the preparation of Elagolix of formula (I) or pharmaceutically acceptable salts, 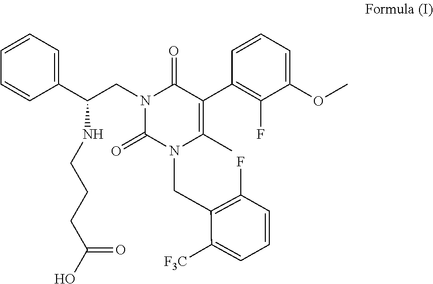 [SMILES: COc1cccc(-c2c(C)n(Cc3c(F)cccc3C(F)(F)F)c(=O)n(C[C@H](NCCCC(=O)O)c3ccccc3)c2=O)c1F]
comprises:
a) reacting the compound of formula (VIa) with 2-flouro3-methoxyphenylboronic acid of formula (VII) in toluene to provide the compound of formula (VIIIa) which is deprotected to produce the compound of formula (IX) 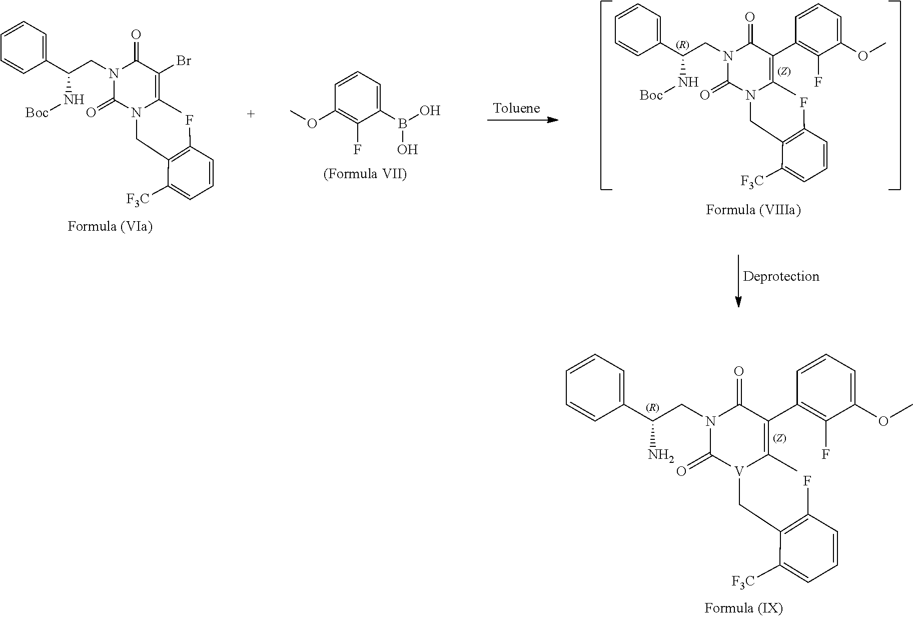
b) converting the compound of formula (IX) to Elagolix or its pharmaceutically acceptable salts.

19. Improved process for the preparation of Elagolix of formula (I) or pharmaceutically acceptable salts, 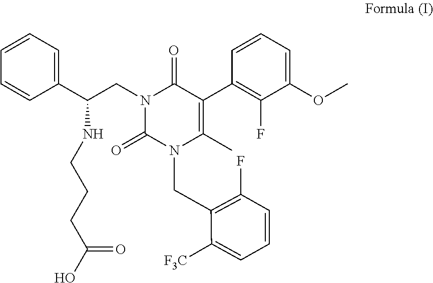 [SMILES: COc1cccc(-c2c(C)n(Cc3c(F)cccc3C(F)(F)F)c(=O)n(C[C@H](NCCCC(=O)O)c3ccccc3)c2=O)c1F]
comprises:
a) reacting the compound of formula (IX) with the compound of formula (X) in presence of a base, phase transfer catalyst in a solvent to provide the compound of formula (XI) 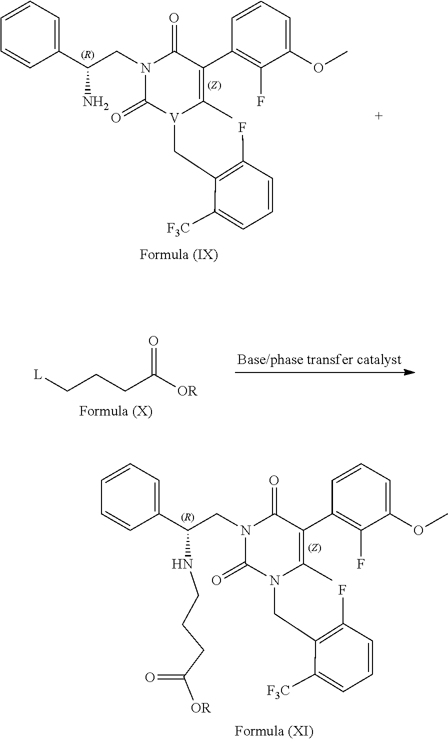
b) converting the compound of formula (XI) to get Elagolix or pharmaceutically acceptable salts
wherein L is a leaving group and R is an alkyl group having C₁-C₄ carbon atoms.

20. The process of claim 19, wherein leaving group is selected from chlorine, bromine, iodine, methanesulfonyloxy (—OMs), toluenesulfonyloxy (—OTs).

21. The process of claim 19, wherein the base used in step-a) is selected from potassium carbonate, sodium carbonate, sodium bicarbonate, potassium hydroxide, potassium carbonate, triethylamine, diisopropylethylamine, ammonia and the like; phase transfer catalyst is selected from quaternary ammonium salts like tetra-butylammonium bromide, tetra-butyl ammonium fluoride, benzyltriethyl ammonium chloride, methyltricapryl ammonium chloride, methyltributyl ammonium chloride, and methyltrioctylammonium chloride, crown ethers, and phosphonium compounds; solvent is selected from hydrocarbon solvents, alcohol solvents, ester solvents, nitrile solvents, chloro solvents, ether solvents or water or mixtures thereof; the conversion in step-b) is carried out by hydrolysis using a base or acid.

22. Improved process for the preparation of Elagolix of formula (I) or pharmaceutically acceptable salts, 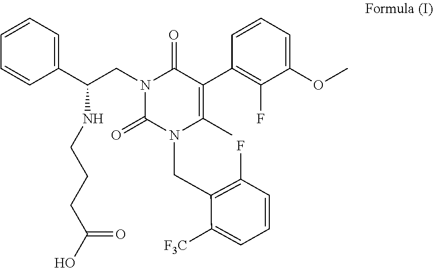 [SMILES: COc1cccc(-c2c(C)n(Cc3c(F)cccc3C(F)(F)F)c(=O)n(C[C@H](NCCCC(=O)O)c3ccccc3)c2=O)c1F]
comprising:
a) reacting the compound of formula (IX) with the compound of formula (X) in presence of K₂CO₃ and tert-butyl ammonium bromide to provide the compound of formula (XIa) 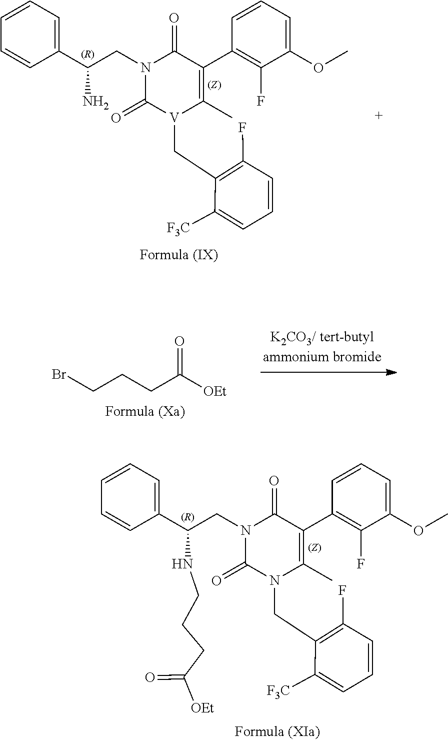
b) converting the compound of formula (XIa) to get Elagolix or pharmaceutically acceptable salts
